(19) United States
(12) Patent Application Publication
(10) Pub. No.: US 2009/0173080 A1
(43) Pub. Date: Jul. 9, 2009

(54) METHOD AND APPARATUS TO FACILITATE SUBSTITUTE NATURAL GAS PRODUCTION

(76) Inventors: Paul Steven Wallace, Katy, TX (US); Arnaldo Frydman, Houston, TX (US)

Correspondence Address: JOHN S. BEULICK (17851);ARMSTRONG TEASDALE LLP, ONE METROPOLITAN SQUARE, SUITE 2600, ST. LOUIS, MO, 63102-2740, US

(21) Appl. No.: 11/970,211

(22) Filed: Jan. 7, 2008

(51) Int. Cl. F02C 3/20; C07C 27/00; C01B 3/32; B01J 8/00

(52) U.S. Cl. 60/780; 518/702; 422/189; 60/39.12; 48/127.1

(57) ABSTRACT

A method of producing substitute natural gas (SNG) includes providing a syngas stream that includes at least some carbon dioxide (CO₂). The method also includes separating at least a portion of the CO₂ from at least a portion of the syngas stream provided. The method further includes channeling at least a portion of the CO₂ separated from at least a portion of the syngas stream to at least a portion of at least one gasification reactor.

### BACKGROUND OF THE INVENTION

The present invention relates generally to integrated gasification combined-cycle (IGCC) power generation plants, and more particularly, to methods and apparatus for optimizing substitute natural gas production and heat transfer with a gasification system.

At least some known IGCC plants include a gasification system that is integrated with at least one power-producing turbine system. For example, known gasification systems convert a mixture of fuel, air or oxygen, steam, and/or carbon dioxide (CO₂) into a synthetic gas, or “syngas”. The syngas is channeled to the combustor of a gas turbine engine, which powers a generator that supplies electrical power to a power grid. Exhaust from at least some known gas turbine engines is supplied to a heat recovery steam generator (HRSG) that generates steam for driving a steam turbine. Power generated by the steam turbine also drives an electrical generator that provides electrical power to the power grid.

At least some known gasification systems associated with IGCC plants produce a syngas fuel for gas turbine engines which is primarily carbon monoxide (CO) and hydrogen (H₂). This syngas fuel typically needs a higher mass flow than natural gas to obtain a similar heat release compared to natural gas. This additional mass flow may require significant turbine modifications and is not directly compatible with standard natural gas-based gas turbines.

Moreover, to facilitate controlling NOₓ emissions during turbine engine operation, at least some known gas turbine engines use combustors that operate with a lean fuel/air ratio, and/or are operated such that fuel is premixed with air prior to being admitted into the combustor's reaction zone. Premixing may facilitate reducing combustion temperatures and subsequently reduce NOₓ formation without requiring diluent addition. However, if the fuel used is a syngas fuel, the syngas fuel selected may include sufficient hydrogen (H₂) such that an associated high flame speed may facilitate autoignition, flashback, and/or flame holding within a mixing apparatus. Moreover, such high flame speed may not facilitate uniform fuel and air mixing prior to combustion. Furthermore, at least one inert diluent, including, but not limited to, nitrogen (N₂), may need to be added into the H₂-rich fuel gas system to prevent excessive NOₓ formation and to control flame autoignition, flashback, and/or flame holding. However, inert diluents are not always available, may adversely affect an engine heat rate, and/or may increase capital and operating costs. Steam may be introduced as a diluent, however, steam may shorten a life expectancy of the hot gas path components.

### BRIEF DESCRIPTION OF THE INVENTION

In one aspect, a method of producing substitute natural gas (SNG) is provided. The method includes providing a syngas stream that includes at least some carbon dioxide (CO₂). The method also includes separating at least a portion of the CO₂ from at least a portion of the syngas stream provided. The method further includes channeling at least a portion of the CO₂ separated from at least a portion of the syngas stream to at least a portion of at least one gasification reactor.

In another aspect, a gasification system is provided. The gasification system includes at least one gasification reactor configured to receive carbon dioxide (CO₂) and to generate a gas stream. The system also includes a CO₂ recycling sub-system coupled in flow communication with the gasification reactor. The sub-system includes at least one gas shift reactor configured to generate CO₂ within the gas stream. The sub-system also includes at least one acid gas removal unit (AGRU) configured to remove the CO₂ from the gas stream. The sub-system further includes at least one conduit to facilitate channeling the CO₂ from the at least one AGRU to the at least one gasification reactor.

In a further aspect, an integrated gasification combined-cycle (IGCC) power generation plant is provided. The IGCC plant includes at least one gas turbine engine coupled in flow communication with at least one gasification system. The gasification system includes at least one gasification reactor configured to receive carbon dioxide (CO₂) and generate a gas stream. The system also includes a CO₂ recycling sub-system coupled in flow communication with the gasification reactor. The sub-system includes at least one gas shift reactor configured to generate CO₂ within the gas stream. The sub-system also includes at least one acid gas removal unit (AGRU) configured to remove the CO₂ from the gas stream. The sub-system further includes at least one conduit to facilitate channeling the CO₂ from the at least one AGRU to the at least one gasification reactor.

### BRIEF DESCRIPTION OF THE DRAWINGS

FIG. 1 is a schematic diagram of an exemplary integrated gasification combined-cycle (IGCC) power generation plant;

FIG. 2 is a schematic diagram of an exemplary gasification system that can be used with the IGCC power generation plant shown in FIG. 1; and

FIG. 3 is a schematic diagram of an alternative gasification system that can be used with the IGCC power generation plant shown in FIG. 1.

### DETAILED DESCRIPTION OF THE INVENTION

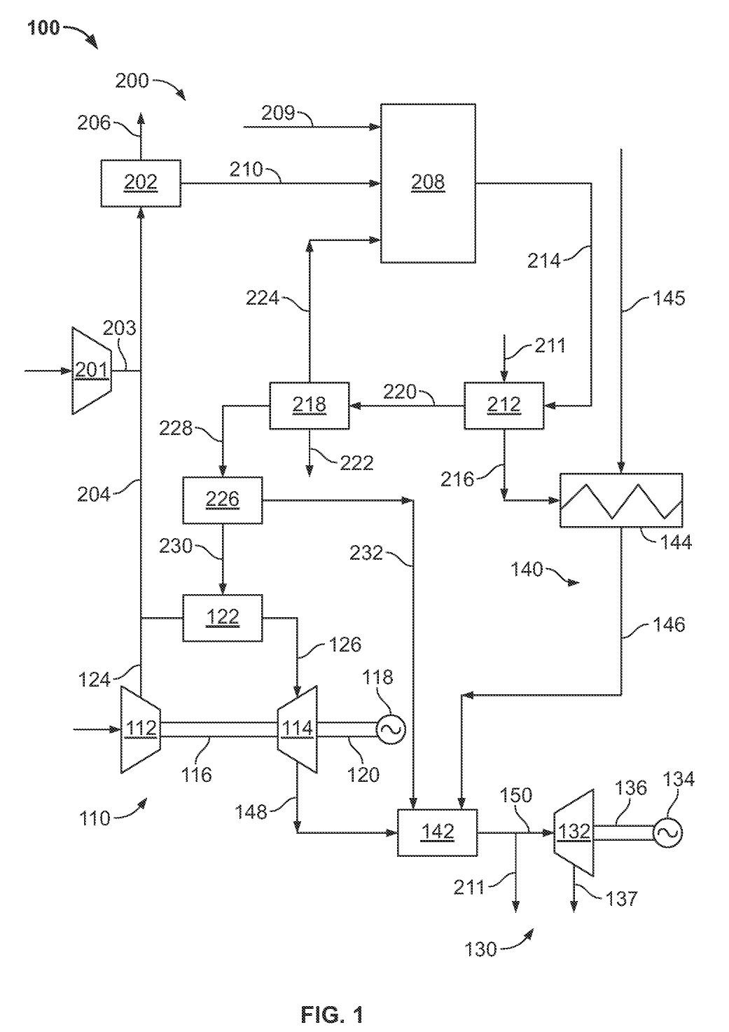

FIG. 1 is a schematic diagram of an exemplary integrated gasification combined-cycle (IGCC) power generation plant 100. In the exemplary embodiment, IGCC plant includes a gas turbine engine 110. Engine 110 includes a compressor 112 rotatably coupled to a turbine 114 via a shaft 116. Compressor 112 is configured to receive air at locally atmospheric pressures and temperatures. Turbine 114 is rotatably coupled to a first electrical generator 118 via a first rotor 120. Engine 110 also includes at least one combustor 122 coupled in flow communication with compressor 112. Combustor 122 is configured to receive at least a portion of air (not shown) compressed by compressor 112 via an air conduit 124. Combustor 122 is also coupled in flow communication with at least one fuel source (described in more detail below) and is configured to receive the fuel from the fuel source. The air and fuel are mixed and combusted within combustor 122 and combustor 122 facilitates production of hot combustion gases (not shown). Turbine 114 is coupled in flow communication with combustor 122 and turbine 114 is configured to receive the hot combustion gases via a combustion gas conduit 126. Turbine 114 is also configured to facilitate converting the heat energy within the gases to rotational energy. The rotational energy is transmitted to generator 118 via rotor 120, wherein generator 118 is configured to facilitate converting the rotational energy to electrical energy (not shown) for transmission to at least one load, including, but not limited to, an electrical power grid (not shown).

IGCC plant 100 also includes a steam turbine engine 130. In the exemplary embodiment, engine 130 includes a steam turbine 132 rotatably coupled to a second electrical generator 134 via a second rotor 136.

IGCC plant 100 further includes a steam generation system 140. In the exemplary embodiment, system 140 includes at least one heat recovery steam generator (HRSG) 142 that is coupled in flow communication with at least one heat transfer apparatus 144 via at least one heated boiler feedwater conduit 146. Apparatus 144 is configured to receive boiler feedwater from a conduit 145. HRSG 142 is also coupled in flow communication with turbine 114 via at least one conduit 148. HRSG 142 is configured to receive boiler feedwater (not shown) from apparatus 144 via conduit 146 for facilitating heating the boiler feedwater into steam. HRSG 142 is also configured to receive exhaust gases (not shown) from turbine 114 via an exhaust gas conduit 148 to further facilitate heating the boiler feedwater into steam. HRSG 142 is coupled in flow communication with turbine 132 via a steam conduit 150.

Conduit 150 is configured to channel steam (not shown) from HRSG 142 to turbine 132. Turbine 132 is configured to receive the steam from HRSG 142 and convert the thermal energy in the steam to rotational energy. The rotational energy is transmitted to generator 134 via rotor 136, wherein generator 134 is configured to facilitate converting the rotational energy to electrical energy (not shown) for transmission to at least one load, including, but not limited to, the electrical power grid. The steam is condensed and returned as boiler feedwater via a condensate conduit 137.

IGCC plant 100 also includes a gasification system 200. In the exemplary embodiment, system 200 includes at least one air separation unit 202 coupled in flow communication with compressor 112 via an air conduit 204. Air separation unit is also coupled in flow communication with at least one compressor 201 via an air conduit 203 wherein compressor 201 is configured to supplement compressor 112. Alternatively, air separation unit 202 is coupled in flow communication to air sources that include, but are not limited to, dedicated air compressors and compressed air storage units (neither shown). Unit 202 is configured to separate air into oxygen (O₂) and other constituents (neither shown). The other constituents are released via vent 206.

System 200 includes a gasification reactor 208 that is coupled in flow communication with unit 202 and is configured to receive the O₂ channeled from unit 202 via an O₂ conduit 210. Reactor 208 is also configured to receive coal 209 and to facilitate production of a sour synthetic gas (syngas) stream (not shown).

System 200 also includes a gas shift reactor 212 that is coupled in flow communication with reactor 208 and is configured to receive the sour syngas stream from gasification reactor 208 via sour syngas conduit 214. Reactor 212 is also coupled in flow communication with steam conduit 150 and is further configured to receive at least a portion of the steam channeled from HRSG 142 via a steam conduit 211. Gas shift reactor 212 is further configured to facilitate production of a shifted sour syngas stream (not shown) that includes carbon dioxide (CO₂) and hydrogen (H₂) at increased concentrations as compared to the sour syngas stream produced in reactor 208. In the exemplary embodiment, reactor 212 is also coupled in heat transfer communication with heat transfer apparatus 144 via a heat transfer conduit 216. Conduit 216 is configured to facilitate transferring heat generated within reactor 212 via exothermic chemical reactions associated with shifting the syngas. Apparatus 144 is configured to receive at least a portion of the heat generated within reactor 212. Alternatively, reactor 212 and heat transfer apparatus 144 are consolidated into a single piece of equipment (not shown).

System 200 further includes an acid gas removal unit (AGRU) 218 that is coupled in flow communication with reactor 212 and is configured to receive the shifted sour syngas stream with the increased CO₂ and H₂ concentrations from reactor 212 via a shifted sour syngas conduit 220. AGRU 218 is also configured to facilitate removal of at least a portion of acid components (not shown) from the sour shifted syngas stream via an acid conduit 222. AGRU 218 is further configured to facilitate removal of at least a portion of the CO₂ contained in the sour shifted syngas stream. AGRU 218 is also configured to facilitate producing a sweetened syngas stream (not shown) from at least a portion of the sour syngas stream. AGRU 218 is coupled in flow communication with reactor 208 via a CO₂ conduit 224 wherein a stream of CO₂ (not shown) is channeled to predetermined portions of reactor 208 (discussed further below).

System 200 also includes a methanation reactor 226 that is coupled in flow communication with AGRU 218 and is configured to receive the sweetened syngas stream from AGRU 218 via a sweetened syngas conduit 228. Reactor 226 also is configured to facilitate producing a substitute natural gas (SNG) stream (not shown) from at least a portion of the sweetened syngas stream. Reactor 226 is also coupled in flow communication with combustor 122 wherein the SNG stream is channeled to combustor 122 via a SNG conduit 230. Moreover, reactor 226 is coupled in heat transfer communication with HRSG 142 via a heat transfer conduit 232. Such heat transfer communication facilitates transfer of heat to HRSG 142 that is generated by the sweetened syngas-to-SNG conversion process performed within reactor 226.

In operation, compressor 201 receives atmospheric air, compresses the air and channels the compressed air to air separation unit 202 via conduits 203 and 204. Unit 202 may also receive air from compressor 112 via conduits 124 and 204. The compressed air is separated into O₂ and other constituents. The other constituents are vented via vent 206 and the O₂ is channeled to gasification reactor 208 via conduit 210. Reactor 208 receives the O₂ via conduit 210, coal 209, and CO₂ from AGRU 218 via conduit 224. Reactor 208 facilitates production of a sour syngas stream that is channeled to gas shift reactor 212 via a conduit 214. Steam is channeled to reactor 212 from HRSG 142 via conduits 150 and 211. The sour syngas stream is used to produce the shifted sour syngas stream via exothermic chemical reactions. The shifted syngas stream includes CO₂ and H₂ at increased concentrations as compared to the sour syngas stream produced in reactor 208. The heat from the exothermic reactions is channeled to heat transfer apparatus 144 via heat transfer conduit 216.

Moreover, in operation, the shifted syngas stream is channeled to AGRU 218 via conduit 220 wherein acid constituents are removed via conduit 222 and CO₂ is channeled to reactor 208 via conduit 224. In this manner, AGRU 218 produces a sweetened syngas stream that is channeled to methanation reactor 226 via channel 228 wherein the SNG stream is produced from the sweetened syngas stream via exothermic chemical reactions. The heat from the reactions is channeled to HRSG 142 via conduit 232 and the SNG stream is channeled to combustor 122 via conduit 230.

Further, in operation, turbine 114 rotates compressor 112 such that compressor 112 receives and compresses atmospheric air and channels a portion of the compressed air to unit 202 and a portion to combustor 122. Combustor 122 mixes and combusts the air and SNG and channels the hot combustion gases to turbine 114. The hot gases induce rotation of turbine 114 which subsequently rotates first generator 118 via rotor 120 as well as compressor 112.

At least a portion of the combustion gases are channeled from turbine 114 to HRSG 142 via conduit 148. Also, the at least a portion of the heat generated in reactor 226 is channeled to HRSG 142 via conduit 232. Moreover, at least a portion of the heat produced in reactor 212 is channeled to heat transfer apparatus 144. Boiler feedwater is channeled to apparatus 144 via conduit 145 wherein the water receives at least a portion of the heat generated within reactor 212. The warm water is channeled to HRSG 142 via conduit 146 wherein the heat from reactor 226 and exhaust gas conduit 148 boils the water to form steam. The steam is channeled to steam turbine 132 and induces a rotation of turbine 132. Turbine 132 rotates second generator 134 via second rotor 136. At least a portion of the steam is channeled to reactor 212 via conduit 211. The steam condensed by turbine 132 is recycled for further use via conduit 137.

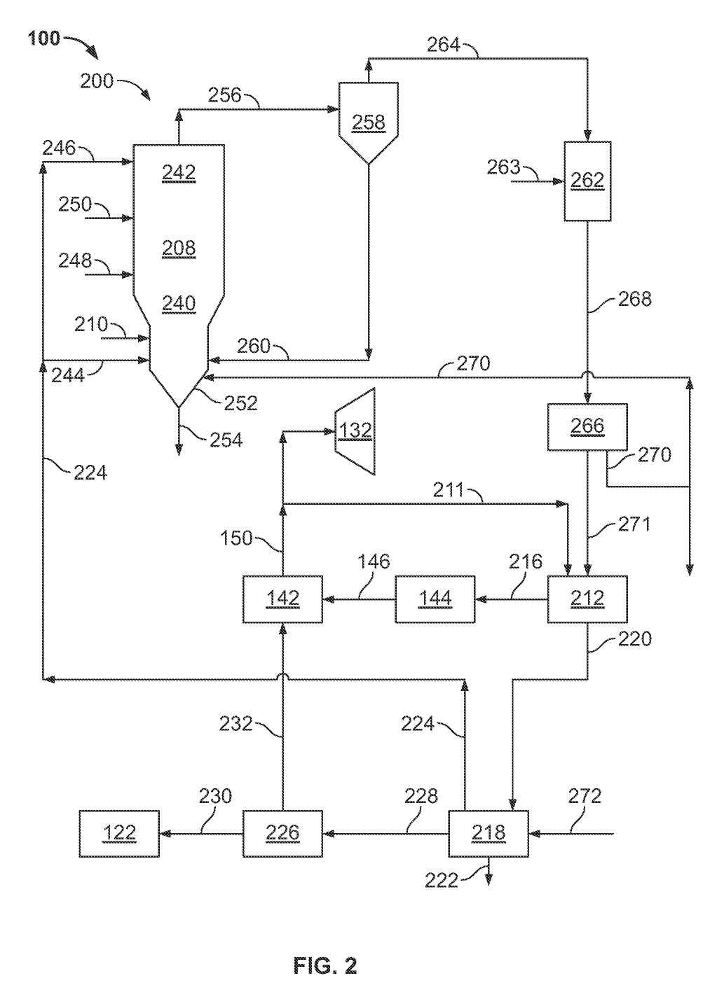

FIG. 2 is a schematic diagram of exemplary gasification system 200 that can be used with IGCC power generation plant 100. System 200 includes gasification reactor 208. Reactor 208 includes a lower stage 240 and an upper stage 242. In the exemplary embodiment, lower stage 240 receives O₂ via conduit 210 such that lower stage 240 is coupled in flow communication with air separation unit 202 (shown in FIG. 1).

CO₂ conduit 224 is coupled in flow communication with a lower stage CO₂ conduit 244 and an upper stage CO₂ conduit 246. As such, lower stage 240 and upper stage 242 are coupled in flow communication to AGRU 218. Moreover, lower stage 240 and upper stage 242 receive dry coal via a lower coal conduit 248 and an upper coal conduit 250, respectively.

Lower stage 240 includes a lock hopper 252 that temporarily stores liquid slag received from lower stage 240. In the exemplary embodiment, hopper 252 is filled with water. Alternatively, hopper 252 has any configuration that facilitates operation of system 200 as described herein. The slag is removed via a conduit 254. Upper stage 242 facilitates removal of a char-laden, sour, hot syngas stream (not shown) via a removal conduit 256. Conduit 256 couples gasification reactor 208 in flow communication with a separator 258. Separator 258 separates sour, hot syngas from the char, such that the char may be recycled back to lower stage 240 via a return conduit 260. In the exemplary embodiment, separator 258 is a cyclone-type separator. Alternatively, separator 258 is any type of separator that facilitates operation of system 200 as described herein.

Separator 258 is coupled in flow communication with a quenching unit 262 via a conduit 264. Quenching unit 262 adds and mixes water (channeled via a conduit 263) with the sour, hot syngas stream in conduit 264 to facilitate cooling of the hot syngas stream, such that a sour, quenched syngas stream (not shown) is formed. Quenching unit 262 is coupled in flow communication with a fines removal unit 266 via a conduit 268. In the exemplary embodiment, unit 266 is a filtration-type unit. Alternatively, unit 266 is any type of unit that facilitates operation of system 200 as described herein including, but not limited to, a water scrubbing-type unit. The fines removed from the sour, quenched syngas stream are channeled to a fines removal unit (not shown) via a removal conduit 270. Unit 266 is also coupled in flow communication with gas shift reactor 212 via a conduit 271.

Reactor 212 is coupled in flow communication with steam conduit 150 and receives at least a portion of steam channeled from HRSG 142 via conduit 211. Reactor 212 is also coupled in heat transfer communication with heat transfer apparatus 144 via conduit 216. Conduit 216 facilitates transferring heat generated within reactor 212 via exothermic chemical reactions associated with shifting the syngas. Apparatus 144 receives at least a portion of the heat generated within reactor 212. HRSG 142 is coupled in flow communication with heat transfer apparatus 144 via heated boiler feedwater conduit 146. Gas shift reactor 212 also facilitates production of a shifted sour syngas stream (not shown) that includes CO₂ and H₂ at increased concentrations as compared to the sour syngas stream produced in reactor 208.

AGRU 218 is coupled in flow communication with reactor 212 and receives the shifted sour syngas stream with the increased CO₂ and H₂ concentrations from reactor 212 via conduit 220. AGRU 218 also facilitates removal of at least a portion of acid components (not shown) that include, but are not limited to, sulfuric and carbonic acids, from the sour shifted syngas stream via conduit 222. To further facilitate acid removal, AGRU 218 receives a solvent that includes, but is not limited to, amine, methanol, and/or Selexol® via a conduit 272. Such acid removal thereby facilitates producing a sweetened syngas stream (not shown) from the sour syngas stream. AGRU 218 also facilitates removal of at least a portion of the gaseous CO₂ contained in the sour shifted syngas stream. Moreover, AGRU 218 is coupled in flow communication with reactor 208 via conduit 224 such that a stream of CO₂ (not shown) is channeled to reactor 208 lower stage 240 and upper stages 242 via conduits 244 and 246, respectively.

Methanation reactor 226 is coupled in flow communication with AGRU 218 and receives the sweetened syngas stream from AGRU 218 via conduit 228. Reactor 226 facilitates producing a substitute natural gas (SNG) stream (not shown) from at least a portion of the sweetened syngas stream. Reactor 226 is also coupled in flow communication with combustor 122 such that the SNG stream is channeled to combustor 122 via conduit 230. Moreover, reactor 226 is coupled in heat transfer communication with HRSG 142 via conduit 232 to facilitate a transfer of heat to HRSG 142 that is generated by the sweetened syngas-to-SNG conversion process performed within reactor 226.

An exemplary method of producing substitute natural gas (SNG) includes providing a syngas stream that includes at least some carbon dioxide (CO2). The method also includes separating at least a portion of the CO₂ from at least a portion of the syngas stream provided. The method further includes channeling at least a portion of the CO₂ separated from at least a portion of the syngas stream to at least a portion of gasification reactor 208.

During operation, O₂ from separator unit 202 and preheated coal are introduced into lower stage 240 via conduits 210 and 248, respectively. The coal and the O₂ are reacted with preheated char introduced into lower stage 240 via conduit 260 to produce a syngas containing primarily H₂, CO, CO₂ and at least some hydrogen sulfide (H₂S). Such syngas formation is via chemical reactions that are substantially exothermic in nature and the associated heat release generates operational temperatures within a range of approximately 1371 degrees Celsius (° C.) (2500 degrees Fahrenheit (° F.)) to approximately 1649° C. (3000° F.). At least some of the chemical reactions that form syngas also form a slag (not shown). The high temperatures within lower stage 240 facilitate maintaining a low viscosity for the slag such that substantially most of the liquid slag can be gravity fed into hopper 252 wherein the relatively cool water in hopper 252, facilitates rapid quenching and breaking of the slag. The syngas flows upward through reactor 208 wherein, through additional reactions in upper stage 242, some of the slag is entrained. In the exemplary embodiment, the coal introduced into lower stage 240 is a dry, or low-moisture, coal that is pulverized to a sufficient particle size to permit entrainment of the pulverized coal with the synthesis gas flowing from lower stage 240 to upper stage 242.

In the exemplary embodiment, CO₂ from AGRU 218 is introduced into lower stage 240 via conduits 224 and 244. The additional CO₂ facilitates increasing an efficiency of IGCC plant 100 by decreasing the required mass flow rate of O₂ introduced via conduit 210. The O₂ molecules from conduit 210 are supplanted with O₂ molecules formed by the dissociation of CO₂ molecules into their constituent carbon (C) and O₂ molecules. As such, additional air for combustion within turbine engine combustor 122 is available for a predetermined compressor rating, thereby facilitating gas turbine engine 110 operating at or beyond rated power generation. Moreover, IGCC plant 100 efficiency is increased since steam from HRSG 142 is not needed to supply O₂ molecules via the dissociation of the steam into H₂ and O₂ molecules. More specifically, the supplanted steam is available for use within steam turbine engine 130, thereby facilitating steam turbine engine 130 operating at or beyond rated power generation. Furthermore, reducing the need for the injection of steam into reactor 208 substantially eliminates the associated loss of heat energy within reactor 208 due to the steam's heat of vaporization properties. Therefore, lower stage 240 operates at a relatively higher efficiency as compared to some known gasification reactors.

The chemical reactions conducted in upper stage 242 are conducted at a temperature in a range of approximately 816° C. (1500° F.) to approximately 982° C. (1800° F.) and at a pressure in excess of approximately 30 bars, or 3000 kiloPascal (kPa) (435 pounds per square inch (psi)) with a sufficient residence time that facilitates the reactants in upper stage 242 reacting with the coal. Moreover, additional dry, preheated coal and CO₂ are introduced into upper stage 242 via conduits 250 and 246, respectively. The syngas and other constituents that rise from lower stage 240, and the additional coal and CO₂ are mixed together to form exothermic chemical reactions that also form steam, char, methane (CH₄) and other gaseous hydrocarbons (including C2+, or, hydrocarbon molecules with at least two carbon atoms). The C2+ hydrocarbon molecules and a portion of the CH₄ reacts with the steam and CO₂ to form a hot, char-laden syngas stream. The temperature range of upper stage 242 is predetermined to facilitate formation of CH₄ and mitigate formation of C2+ hydrocarbon molecules.

At least one product of the chemical reactions within upper stage 242, i.e., between the preheated coal and the syngas, is a low-sulfur char that is entrained in the hot, sour syngas containing CH₄, H₂, CO, CO₂ and at least some H₂S. The sulfur content of the char is maintained at a minimum level by reacting the pulverized coal with the syngas in the presence of H₂ and steam at elevated temperatures and pressures.

The low-sulfur char and liquid slag that are entrained in the hot, sour synthesis gas stream are withdrawn from upper stage 242 and is channeled through conduit 256 into separator 258. A substantial portion of the char and slag are separated from the hot, sour syngas stream in separator 258 and are withdrawn therefrom. The char and slag are channeled through conduit 260 into lower stage 240 for use as a reactant and for disposal, respectively.

The hot, sour syngas is channeled from separator 258 through conduit 264 to quenching unit 262. Quenching unit 262 facilitates removal of any remaining char and slag within the syngas stream. Water is injected into the syngas stream via conduit 263 wherein the entrained char and slag are rapidly cooled and embrittled to facilitate breakage of the slag and char into fines. The water is vaporized and the heat energy associated with the water's latent heat of vaporization is removed from the hot, sour syngas stream and the syngas stream temperature is decreased to approximately 900° C. (1652° F.). The steam entrained within the hot, sour syngas stream is used in subsequent gas shift reactions (described below) with a steam-to-dry gas ratio of approximately 0.8-0.9. The syngas stream with the entrained steam, char, and slag is channeled to fines removal unit 266 via conduit 268 wherein the char and slag fines are removed. In the exemplary embodiment, the char and slag fines are channeled into lower stage 240 for use as a reactant and for disposal, respectively, via conduit 270. Alternatively, the char and slag fines are channeled to a collection unit (not shown) for disposal.

The hot, sour, steam-laden syngas stream is channeled from unit 266 to gas shift reactor 212 via conduit 271. Reactor 212 facilitates formation of CO₂ and H₂ from the CO and H₂O (in the form of steam) within the syngas stream via an exothermic chemical reaction:

CO+H₂O [symbol] CO₂+H₂ (1)

Moreover, heat is transferred from the hot, syngas stream into boiler feedwater via conduit 216 and heat transfer apparatus 144. In the exemplary embodiment, conduit 216 and heat transfer apparatus 144 are configured within reactor 212 as, but not limited to, a shell and tube-type heat exchanger. Alternatively, conduit 216 and apparatus 144 have any configuration that facilitates operation of IGCC plant 100 as described herein. The heated boiler feedwater is channeled to HRSG 142 via conduit 146 for conversion into steam (described below in more detail). Therefore, the hot, sour syngas stream that is channeled into reactor 212 is cooled from approximately 900° C. (1652° F.) to a temperature above approximately 371° C. (700° F.) and is shifted to a cooled, sour syngas stream with an increased concentration of CO₂ and H₂ and with a steam-to-dry gas ratio of less than approximately 0.2-0.5, and with a H₂-to-CO ratio of at least approximately 3.0. Therefore, sufficient H₂ is available from the original gasification process and the subsequent water gas shift process to meet a stoichiometric requirement of the methanation reaction to occur in reactor 226 wherein there is a three-to-one ratio of H₂ molecules to CO molecules (described below in more detail).

The shifted, cooled, sour syngas stream is channeled from reactor 212 to AGRU 218 via conduit 220. AGRU 218 primarily facilitates removing H₂S and CO₂ from the syngas stream channeled from reactor 212. The H₂S mixed with the syngas stream contacts a selective solvent within AGRU 218. In the exemplary embodiment, the solvent used in AGRU 218 is an amine. Alternatively, the solvent includes, but is not limited to including, methanol, and/or Selexol®. The solvent is channeled to AGRU 218 via solvent conduit 272. A concentrated H₂S stream is withdrawn from the bottom of AGRU 218 via conduit 222 to a recovery unit (not shown) associated with further recovery processes. In addition, CO₂ in the form of carbonic acid is also removed and disposed of in a similar manner. Moreover, gaseous CO₂ is collected within AGRU 218 and is channeled to reactor 208 via conduit 224.

The methods of collecting and recycling CO₂ as described herein facilitate an effective method of CO₂ separation. Moreover, such methods facilitate increasing the throughput of gasification reactor 208 due to the increased O₂ injection into reactor 208.

The sweetened syngas stream is channeled from AGRU 218 to methanation reactor 226 via conduit 228. The sweetened syngas stream is substantially free of H₂S and CO₂ and includes proportionally increased concentrations of CH₄ and H₂. The syngas stream also includes a stoichiometric amount of H₂ necessary to completely convert the CO to CH₄ that is at least 3:1 with respect to the H₂/CO ratio. In the exemplary embodiment, reactor 226 uses at least one catalyst known in the art to facilitate an exothermic chemical reaction such as:

CO+3H₂ [symbol] CH₄+H₂O. (2)

The H₂ in reactor 226 converts at least approximately 95% of the remaining CO to CH₄ such that a SNG stream is channeled to combustor 122 via conduit 230 containing over 90% CH₄ and less than 0.1% CO by volume.

The SNG produced as described herein facilitates the use of dry low NOₓ combustors within gas turbine 110 while reducing a need for diluents. Moreover, such SNG production facilitates using existing gas turbine models with little modification to affect efficient combustion. Furthermore, such SNG increases a safety margin in comparison to fuels having higher H₂ concentrations.

The heat generated in the exothermic chemical reactions within reactor 226 is transferred to HRSG 142 via conduit 232 to facilitate boiling of the feedwater that is channeled to HRSG 142 via conduit 146. The steam being generated is channeled to turbine 132 via conduit 150. Such heat generation has the benefit of improving the overall efficiency of IGCC plant 100. Moreover, the increased temperature of the SNG facilitates an improved efficiency of combustion within combustor 122. In the exemplary embodiment, reactor 226 and conduit 232 are configured within HRSG 142 as, but not limited to, a shell and tube-type heat exchanger. Alternatively, conduit 232, reactor 226 and HRSG 142 have any configuration that facilitates operation of IGCC plant 100 as described herein.

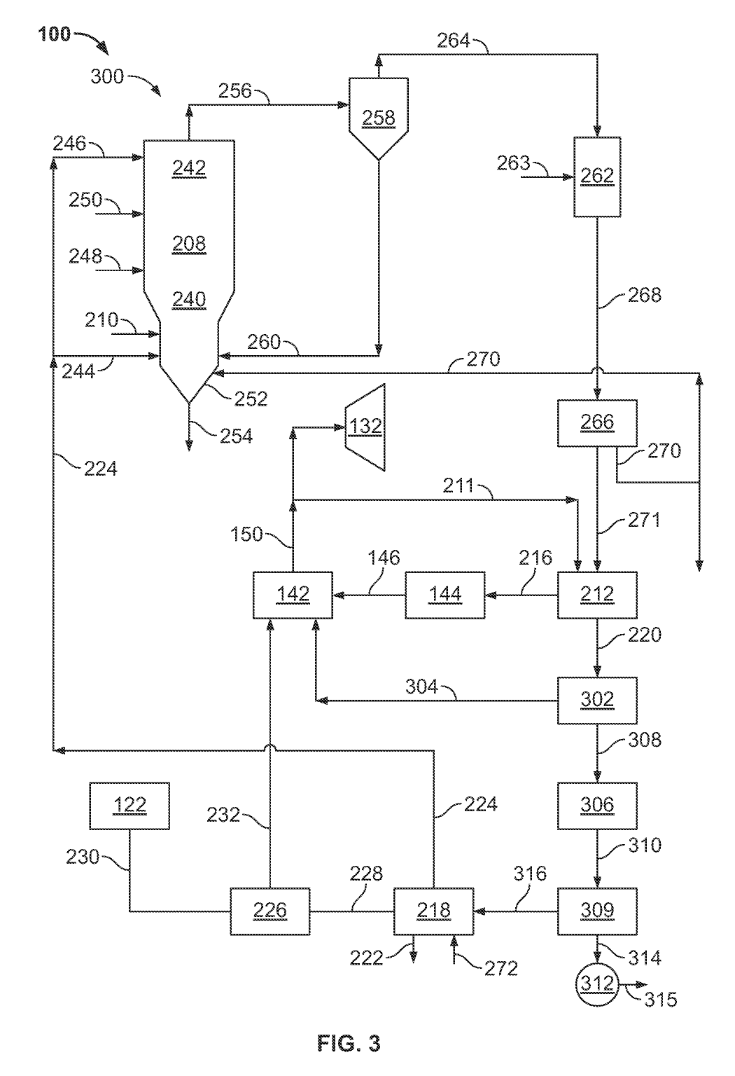

FIG. 3 is a schematic diagram of an alternative gasification system 300 that can be used with IGCC power generation plant 100. System 300 is substantially similar to system 200 (shown in FIG. 2) from reactor 208 to reactor 212 as described above.

System 300 includes a cooled methanation reactor 302 that is coupled in flow communication with reactor 212 and receives the shifted sour syngas stream with the increased CO₂ and hydrogen H₂ concentrations from reactor 212 via conduit 220. Reactor 302 is similar to reactor 226 as described above. Reactor 302 also facilitates producing a partially methanated syngas stream (not shown) from at least a portion of the shifted sour syngas stream. Moreover, reactor 302 is coupled in heat transfer communication with HRSG 142 via a conduit 304. Such heat transfer communication facilitates transfer of heat to HRSG 142 that is generated by the sour syngas-to-partially-methanated syngas conversion process performed within reactor 302. In this alternative embodiment, reactor 302 and conduit 304 are contained within HRSG 142 and are configured as, but not limited to, a shell and tube-type heat exchanger. Alternatively, conduit 304, reactor 302 and HRSG 142 have any configuration that facilitates operation of IGCC plant 100 as described herein. In the exemplary embodiment, reactor 302 is also coupled in flow communication with heat transfer apparatus 306 wherein the partially-methanated syngas stream is channeled to apparatus 306 via a conduit 308. Alternatively, reactor 302 and heat transfer apparatus 306 are consolidated into a single piece of equipment (not shown).

Apparatus 306 receives the partially-methanated syngas stream and transfers at least a portion of the heat contained therein to the boiler feedwater. Apparatus 306 also partially heats the boiler feedwater prior to the water being channeled to HRSG 142. In this alternative embodiment, at least one of either heat transfer apparatus 144 and apparatus 306 is equivalent to a boiler economizer as is known in the art. Therefore, either apparatus 144 or 306 is equivalent to a boiler feedwater heater as is known in the art. Selection of which of apparatus 144 and 306 is an economizer depends upon factors that include, but are not limited to, the heat content of the associated inlet fluids.

Apparatus 306 is coupled in flow communication with a trim cooler 309 via a conduit 310. Cooler 308 is configured to cool the partially-methanated syngas stream channeled from apparatus 306 and to remove a significant portion of the remaining latent heat of vaporization such that the steam within the syngas stream is condensed. Cooler 309 is coupled in flow communication with a knockout drum 312 via conduit 314. Knockout drum 312 is also coupled in flow communication with a condensate recycling system (not shown) via conduit 315. Cooler 309 is coupled in flow communication with AGRU 218 via a conduit 316 wherein the remaining portions of system 300 are substantially similar to the associated equivalents in system 200.

During operation, system 300, up to and including reactor 212, forms the shifted, sour syngas stream as described above. The syngas stream includes an increased concentration of CO₂ and H₂ with a steam-to-dry gas ratio of less than approximately 0.2-0.5 and with a H₂-to-CO ratio of at least approximately 3.0. Therefore, sufficient H₂ is available to meet the stoichiometric requirement of the methanation reaction wherein there is a three-to-one ratio of H₂ molecules to CO molecules.

In the exemplary embodiment, the shifted, sour syngas stream is channeled from reactor 212 to methanation reactor 302 via conduit 220. Reactor 302 facilitates at least partial conversion of the CO to CH₄ in a manner similar to that in reactor 226. The H₂ in reactor 302 converts a approximately 80% to 90% of the CO to H₂O and CH₄. The heat generated in the exothermic chemical reactions within reactor 302 is transferred to HRSG 142 via conduit 304 to facilitate boiling to steam the feedwater that is channeled to HRSG 142. Such heat generation has the benefit of improving the overall efficiency of IGCC plant 100. Alternatively, reactors 212 and 302 are consolidated into a single piece of equipment (not shown), wherein a water-gas shift portion is upstream of a methanation portion, and conduit 220 is eliminated.

A hot, sour, shifted syngas stream (not shown) produced within reactor 302 is channeled to heat transfer apparatus 306 via conduit 308. The heat contained within the syngas stream is transferred to the boiler feedwater via apparatus 306 to facilitate improving the overall efficiency of IGCC plant 100. A cooled, sour, shifted syngas stream is channeled from apparatus 306 to trim cooler 309. Trim cooler 309 facilitates removing at least some of the remaining latent heat of vaporization from the syngas stream such that a substantial portion of the remaining H₂O is condensed and removed from the syngas stream via knockout drum 312. The condensate (not shown) is channeled from drum 312 to the condensate recycling system for reuse with quenching unit 262 and/or fines removal unit 266.

A substantially dry, cooled, sour, and partially-methanated syngas stream (not shown) is channeled to AGRU 218 via conduit 316. In the exemplary embodiment, channeling such a syngas stream to AGRU 218 facilitates using a refrigerated lean oil acid gas removal process as is known in the art in place of or in addition to the amine-related process as described above. Using a refrigerated lean oil process facilitates reducing the use of amines, thereby facilitating a reduction in plant 100 operating costs. Such use also facilitates a reduction in the production of heat stable salt production that is typically associated with using amines for acid gas removal. Such heat stable salts may facilitate production of additional corrosive acids and may reduce the effectiveness of the amines to effective remove the acid within the syngas stream.

Alternatively, channeling such a syngas stream to AGRU 218 facilitates using a natural gas sweetening membrane system as is known in the art in place of or in addition to the amine-related process as described above. Using a membrane system for bulk separation facilitates reducing the use of amines, thereby facilitating a reduction in plant 100 operating costs.

The SNG stream channeled to combustor 122 is produced substantially as described above with the exception that reactor 226 converts the remaining CO and H₂ in the partially-methanated syngas stream to produce CH₄ and H₂O as described above.

The method and apparatus for substitute natural gas, or SNG, production as described herein facilitates operation of integrated gasification combined-cycle (IGCC) power generation plants, and specifically, SNG production systems. More specifically, collecting and recycling carbon dioxide (CO₂) molecules within the SNG production system facilitates a method of CO₂ separation. Also specifically, configuring the IGCC and SNG production systems as described herein facilitates optimally generating and collecting heat from the exothermic chemical reactions in the SNG production process to facilitate improving IGCC plant thermal efficiency. Moreover, the method and equipment for producing such SNG as described herein facilitates retrofitting existing in-service gas turbines by reducing hardware modifications as well as reducing capital and labor costs associated with affecting such modifications.

Exemplary embodiments of SNG production as associated with IGCC plants are described above in detail. The methods, apparatus and systems are not limited to the specific embodiments described herein nor to the specific illustrated IGCC plants.

While the invention has been described in terms of various specific embodiments, those skilled in the art will recognize that the invention can be practiced with modification within the spirit and scope of the claims.

What is claimed is:

1. A method of producing substitute natural gas (SNG), said method comprising:
providing a syngas stream that includes at least some carbon dioxide (CO₂);
separating at least a portion of the CO₂ from at least a portion of the syngas stream provided; and
channeling at least a portion of the CO₂ separated from at least a portion of the syngas stream to at least a portion of at least one gasification reactor.

2. A method in accordance with claim 1 wherein providing a syngas stream that includes at least some CO₂ comprises:
producing a syngas stream with the at least one gasification reactor;
channeling at least a portion of the syngas stream to at least one gas shift reactor; and
producing a shifted syngas stream that includes at least some carbon dioxide (CO₂) in the at least one gas shift reactor.

3. A method in accordance with claim 2 wherein producing a shifted syngas stream comprises transferring heat from at least a portion of the at least one gas shift reactor via at least one heat transfer apparatus.

4. A method in accordance with claim 1 wherein separating at least a portion of the CO₂ from at least a portion of the syngas stream comprises:
channeling the shifted syngas stream including at least some CO₂ to at least one acid gas removal unit (AGRU); and
separating at least a portion of the CO₂ from at least a portion of the shifted syngas stream within the at least one AGRU.

5. A method in accordance with claim 4 wherein separating at least a portion of the CO₂ from at least a portion of the shifted syngas stream comprises sequestering at least a portion of the CO₂ from at least a portion of the shifted syngas stream.

6. A method in accordance with claim 1 wherein channeling at least a portion of the CO₂ separated from at least a portion of the syngas stream comprises:
forming at least one CO₂ stream; and
injecting at least a portion of the at least one CO₂ stream into the gasification reactor.

7. A method in accordance with claim 1 further comprising:
producing an SNG stream from at least a portion of a shifted syngas stream within at least one methanation reactor; and
channeling at least a portion of the shifted syngas stream to the at least one methanation reactor from at least one of at least one AGRU and at least one gas shift reactor.

8. A method in accordance with claim 7 wherein producing an SNG stream comprises transferring heat from at least a portion of the at least one methanation reactor via at least one heat transfer apparatus.

9. A method in accordance with claim 1 further comprising coupling at least a portion of a steam generation system in heat transfer communication with at least one of:
at least a portion of at least one gas shift reactor; and
at least a portion of at least one methanation reactor.

10. A gasification system comprising:
at least one gasification reactor configured to receive carbon dioxide (CO₂) and to generate a gas stream; and
a CO₂ recycling sub-system coupled in flow communication with said gasification reactor, said sub-system comprising:
at least one gas shift reactor configured to generate CO₂ within said gas stream;
at least one acid gas removal unit (AGRU) configured to remove the CO₂ from said gas stream; and
at least one conduit to channel the CO₂ from said at least one AGRU to said at least one gasification reactor.

11. A gasification system in accordance with claim 10 wherein said at least one gas shift reactor is coupled in flow communication with said gasification reactor and said AGRU, said at least one gas shift reactor is configured to capture at least a portion of heat released from at least one exothermic chemical reaction, wherein said at least one gas shift reactor is one of:
coupled in heat transfer communication with at least one external heat transfer apparatus; and
consolidated in a unitary enclosure with at least one integrated heat transfer apparatus.

12. A gasification system in accordance with claim 10 further comprising at least one methanation reactor coupled in flow communication with said AGRU, said at least one methanation reactor is configured to capture at least a portion of heat released from at least one exothermic chemical reaction, wherein said at least one methanation reactor is one of:
coupled in heat transfer communication with at least one external heat transfer apparatus; and
consolidated in a unitary enclosure with at least one integrated heat transfer apparatus.

13. A gasification system in accordance with claim 12 wherein said methanation reactor is coupled in flow communication with said gas shift reactor, said at least one methanation reactor is configured to capture at least a portion of heat release from at least one exothermic chemical reaction, wherein said at least one methanation reactor is one of:
coupled in heat transfer communication with at least one external heat transfer apparatus; and
consolidated in a unitary enclosure with at least one integrated heat transfer apparatus.

14. A gasification system in accordance with claim 10 wherein said gas shift reactor is configured as a gas shift reactor portion within an integrated apparatus, said integrated apparatus comprises a methanation reactor portion downstream of said gas shift reactor portion, said methanation reactor portion is configured to capture at least a portion of heat release from at least one exothermic chemical reaction, wherein said at least one methanation reactor portion is one of:
coupled in heat transfer communication with at least one external heat transfer apparatus; and
consolidated in a unitary section of said integrated apparatus with at least one integrated heat transfer apparatus.

15. An integrated gasification combined-cycle (IGCC) power generation plant comprising at least one gas turbine engine coupled in flow communication with at least one gasification system, said at least one gasification system comprising:
at least one gasification reactor configured to receive carbon dioxide (CO₂) and to generate a gas stream; and
a CO₂ recycling sub-system coupled in flow communication with said gasification reactor, said sub-system comprising:
at least one gas shift reactor configured to generate CO₂ within said gas stream;
at least one acid gas removal unit (AGRU) configured to remove the CO₂ from said gas stream; and
at least one conduit to facilitate channeling the CO₂ from said at least one AGRU to said at least one gasification reactor.

16. An IGCC power generation plant in accordance with claim 15 wherein said at least one gas shift reactor is coupled in flow communication with said gasification reactor and said AGRU, said at least one gas shift reactor is configured to capture at least a portion of heat released from at least one exothermic chemical reaction, wherein said at least one gas shift reactor is one of:
coupled in heat transfer communication with at least one external heat transfer apparatus; and
consolidated in a unitary enclosure with at least one integrated heat transfer apparatus.

17. An IGCC power generation plant in accordance with claim 15 further comprising at least one methanation reactor coupled in flow communication with said AGRU, said at least one methanation reactor is configured to capture at least a portion of heat released from at least one exothermic chemical reaction, wherein said at least one methanation reactor is one of:
coupled in heat transfer communication with at least one external heat transfer apparatus; and
consolidated in a unitary enclosure with at least one integrated heat transfer apparatus.

18. An IGCC power generation plant in accordance with claim 17 wherein said methanation reactor is coupled in flow communication with said gas shift reactor, said at least one methanation reactor is configured to capture at least a portion of heat released from at least one exothermic chemical reaction, wherein said at least one methanation reactor is one of:
coupled in heat transfer communication with at least one external heat transfer apparatus; and
consolidated in a unitary enclosure with at least one integrated heat transfer apparatus.

19. An IGCC power generation plant in accordance with claim 15 wherein said at least one gas shift reactor is configured as a gas shift reactor portion within an integrated apparatus, said integrated apparatus comprises a methanation reactor portion downstream of said gas shift reactor portion, said methanation reactor portion is configured to capture at least a portion of heat release from at least one exothermic chemical reaction, wherein said at least one methanation reactor portion is one of:
coupled in heat transfer communication with at least one external heat transfer apparatus; and
consolidated in a unitary section of said integrated apparatus with at least one integrated heat transfer apparatus.

20. An IGCC power generation plant in accordance with claim 15 further comprising a steam generation system coupled in flow communication with at least one steam turbine, said steam generation system is further coupled in heat transfer communication with at least one of:
a portion of said gasification system; and
a portion of said gas turbine engine.
